(19) United States
(12) Patent Application Publication
(10) Pub. No.: US 2026/0002171 A1
(43) Pub. Date: Jan. 1, 2026

(54) METHOD FOR IMPROVING AGRONOMIC TRAITS OF PLANT AND USE THEREOF

(71) Applicants: BIOTECHNOLOGY RESEARCH INSTITUTE OF CHINESE ACADEMY OF AGRICULTURAL SCIENCES, Beijing (CN); SOUTH CHINA AGRICULTURAL UNIVERSITY, Guangzhou (CN); Yazhouwan National Laboratory, Sanya (CN)

(72) Inventors: Haiyang WANG, Guangzhou (CN); Miaoyun XU, Beijing (CN); Lei MA, Beijing (CN); Zhijing BAI, Beijing (CN); Baobao WANG, Beijing (CN); Yurong XIE, Beijing (CN); Binbin ZHAO, Guangzhou (CN); Hua XU, Guangzhou (CN); Zhigang ZHENG, Guangzhou (CN)

(73) Assignees: BIOTECHNOLOGY RESEARCH INSTITUTE OF CHINESE ACADEMY OF AGRICULTURAL SCIENCES, Beijing (CN); SOUTH CHINA AGRICULTURAL UNIVERSITY, Guangzhou (CN); Yazhouwan National Laboratory, Sanya (CN)

(21) Appl. No.: 19/131,550

(22) PCT Filed: Feb. 6, 2023
(86) PCT No.: PCT/CN2023/074545

§ 371 Date: May 21, 2025

(30) Foreign Application Priority Data
Jan. 4, 2023 (CN) 202310005958.9

(51) Int. Cl. C12N 15/82; C07K 14/415

(52) U.S. Cl. CPC C12N 15/8273; C07K 14/415

(57) ABSTRACT

A method for improving agronomic traits of plants and the use thereof are provided. By means of overexpressing functional gene ZmSPL12 in plants, the salt stress resistance, alkali stress resistance and/or resistance to low nitrogen of plants are improved. The method and the use thereof provide the possibility for cultivating new salt-alkali resistant, and low-nitrogen resistant crop varieties, especially new com varieties, and are of great significance for global grain safety and agricultural sustainable development.

### CROSS REFERENCE TO THE RELATED APPLICATIONS

This application is the national phase entry of International Application No. PCT/CN2023/074545, filed on Feb. 6, 2023, which is based upon and claims priority to Chinese Patent Application No. 202310005958.9, filed on Jan. 4, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBYJS070-PKG_Sequence_Listing.xml, created on Apr. 27, 2025, and is 15,533 bytes in size.

### TECHNICAL FIELD

The present application belongs to the technical field of plant genes, and in particular relates to a method and use of a ZmSPL12 gene and its encoded protein for improving the salt-tolerant, alkali-tolerant and/or low nitrogen-tolerant traits of plants.

### BACKGROUND

Soil salinization is one of the main environmental stresses that significantly reduce crop yield and quality worldwide. According to incomplete statistics, approximately 954.38 million hectares of soil worldwide are affected by salinity, which can result in a productivity loss of up to 7-8%. In addition, excessive use of nitrogen fertilizers in agricultural planting production can easily lead to environmental pollution and increase farmers' planting costs.

Maize is an important crop that serves as a food grain, feed, and industrial raw material and is also the crop with the highest production capacity in the world. Its sufficient and stable supply is crucial for ensuring food grain security worldwide. Therefore, it is of great significance for global food grain security and sustainable agricultural development to study the molecular mechanisms of salt-alkali tolerance and low nitrogen tolerance in maize, exploit resources of excellent genes of salt-alkali tolerance and low nitrogen tolerance, and cultivate new maize varieties with salt-alkali tolerance and low nitrogen tolerance.

### SUMMARY

All references described herein are incorporated herein by reference. Unless indicated to the contrary, all technical and scientific terms used herein have the same meaning as commonly understood by those of ordinary skill in the art to which the present application belongs. Unless otherwise indicated, the techniques used or described herein are standard techniques known to those of ordinary skill in the art. The materials, methods and embodiments are for illustration only, not for limitation.

An embodiment of the present application provides a functional gene SPL12 for improving agronomic traits of a plant, which gene can enhance the tolerance of a plant to salt stress, alkali stress, and/or low nitrogen after being overexpressed in the plant.

An embodiment of the present application provides a method for improving agronomic traits of a plant by introducing an over-expressed construct of a functional gene into the plant to enable the plant to acquire a salt-tolerant, alkali-tolerant and/or low nitrogen-tolerant phenotype, where the functional gene has a nucleotide sequence selected from one of the sequences in the following group:
(a) a nucleotide sequence as shown in a gene position number of Zm00001d015410;
(b) a nucleotide sequence as shown in SEQ ID NO: 1 or 2;
(c) a nucleotide sequence encoding an amino acid sequence as shown in SEQ ID NO: 3;
(d) a nucleotide sequence capable of hybridizing with the nucleotide sequence in the sequences of (a) to (c) under a stringent hybridization condition;
(e) a nucleotide sequence having 80% (preferably at least 85%) sequence similarity with any one of the nucleotide sequences of (a) to (d), and enabling a plant to acquire a salt-tolerant, alkali-tolerant and/or low nitrogen-tolerant phenotype after being overexpressed in the plant; or
(f) a nucleotide sequence complementary to any one of the nucleotide sequences of (a) to (e).

Optionally, the “stringent hybridization condition” or “stringent condition” described in the present application means conditions of low ionic strength and high temperature known in that art. Generally, under a stringent condition, the detectable degree of hybridization between a probe and its target sequence is higher than that of hybridization with other sequences (for example, it exceeds the background by at least 2 times). A stringent hybridization condition is sequence-dependent, which will be different under different environmental conditions. Longer sequences hybridize specifically at higher temperatures. By controlling the stringency of hybridization or washing conditions, the target sequence which is 100% complementary to the probe can be identified. More specifically, the stringent conditions are usually selected to be about 5-10° C. lower than the hot melting point (Tm) of the specific sequence at a specified ionic strength pH. Tm is the temperature at which 50% of the probes complementary to the target hybridize to the target sequence in the equilibrium state. Stringent conditions may be those in which the salt concentration is lower than about 1.0 M sodium ion concentration at pH 7.0 to 8.3, usually about 0.01 to 1.0 M sodium ion concentration, and the temperature is at least about 30° C. for short probes (including but not limited to 10 to 50 nucleotides) and at least about 60° C. for long probes (including but not limited to more than 50 nucleotides). Stringent conditions can also be achieved by adding destabilizers such as formamide. For selective or specific hybridization, the positive signal can be at least 2 times that of background hybridization, and optionally 10times that of background hybridization. Exemplary stringent hybridization conditions can be as follows: 50% formamide, 5×SSC and 1% SDS, cultured at 42° C.; or 5×SSC, 1% SDS, cultured at 65° C., washed in 0.2×SSC and washed in 0.1% SDS at 65° C. The washing may be carried out for 5, 15, 30, 60, 120 minutes or longer.

Optionally, the functional gene provided by the embodiments of the present application further includes a homologous gene which has at least 80%, 85%, 90%, 95%, 98% or 99% sequence similarity to the nucleotide sequence of the functional gene with salt tolerance, alkali tolerance and/or low nitrogen tolerance disclosed by the embodiments of the present application, or a homologous gene which has at least 90%, 95% or 98% sequence similarity to the amino acid sequence of the functional gene disclosed by the embodiments of the present application. And the homologous gene enables a plant to acquire a salt-tolerant, alkali-tolerant and/or low nitrogen-tolerant phenotype after being overexpressed in the plant. The homologous gene can be isolated from any plant.

Optionally, the nucleotide sequence of the homologous gene of the functional gene described in the embodiments of the present application can be isolated from any plant, including but not limited to brassica, maize, wheat, sorghum, crambe, sinapsis alba, castor bean, sesame, cottonseed, linseed, soybean, arabidopsis, phaseolus, peanut, alfalfa, oat, rapeseed, barley, oat, Rye, millet, dhurra, triticale, einkorn wheat, Spelt, emmer wheat, flax, Gramma grass, tripsacum, teosinte, festuca, perennial wheatgrass, sugarcane, cranberry, papaya, banana, safflower, oil palm, melon, apple, cucumber, dendrobium, gladiolus, chrysanthemum, Liliaceae, cotton, eucalyptus, sunflower, brassica, sugarbeet, coffee, ornamental plants, pines, etc. Preferably, the plant includes maize, soybean, mustard, millet, wheat, barley, rye, rice, cotton, and sorghum.

The percentage of sequence similarity described in the present application can be obtained by commonly known bioinformatics algorithms, including Myers and Miller algorithm, Needleman-Wunsch global alignment method, Smith-Waterman local alignment method, Pearson and Lipman similarity search method, and Karlin and Altschul algorithm, which are commonly known to those skilled in the art.

Those skilled in the art should know that there is Single Nucleotide Polymorphism (SNP) in the same gene among different varieties of the same plant, i.e., the nucleotide sequence of the same gene often has individual base differences, but there are so many varieties in the same crop that it is impossible for the inventors to list them one by one. The embodiments of the present application only provide the sequences of representative varieties in maize crops. Therefore, those skilled in the art should know that the nucleotide sequences from different varieties that have SNP with the gene protected by the present application and the nucleotide sequence thereof are also within the protection scope of the present application.

Optionally, in the method for improving agronomic traits of a plant provided by the embodiments of the present application, the over-expressed construct further includes a promoter operably linked to the isolated nucleotide sequence of the functional gene and driving excessive expression of the functional gene. “Excessive expression” or “overexpression” described in the present application means that the expression amount of an expression product of a gene in a transgenic plant exceeds that in a normal or non-transgenic plant.

Optionally, the promoter described in the present application can be a constitutive expression promoter, a specific expression promoter or an inducible expression promoter. Specifically, the overexpression promoter includes but is not limited to a cauliflower mosaic virus CaMV 35s promoter (odelletal., nature 313:810-812(1985)), a nopaline synthase (nos) promoter (ebertetal., pnas. 84:5745-5749(1987)), an adh promoter (walkeretal., pnas 84:6624-6628 (1987)), a sucrose synthase promoter (yangetal., pnas. 87:4144-4148(1990)), a maize ubiquitin promoter Ubiquitin (cornejoetal., plantmolbiol. 23:567-581(1993)), a rice Actin1 promoter, etc.; the specific expression promoter includes but is not limited to a root-specific expression promoter and the like, and especially when the functional gene provided in the embodiments of the present application is applied to enhance plant breeding for low nitrogen tolerance, the specific expression in a root has a better effect on the whole plant, as the root is the main organ for nitrogen absorption; the inducible expression promoter includes but is not limited to an abiotic stress-inducible promoter, a biotic stress-inducible promoter and the like, and when this type of promoter is used to drive the functional gene provided in the embodiments of the present application, the stress-resistance gene expression is not necessary to be activated under a normal growth condition of a plant, but only activated when the plant is subjected to stress, thereby reducing the waste of biomass.

Optionally, the method for improving agronomic traits of a plant provided by the embodiments of the present application can be used for improving the salt-tolerant, alkali-tolerant and/or low nitrogen-tolerant traits of any plant. The plant includes monocotyledons and/or dicotyledons, optionally including but not limited to brassica, maize, wheat, sorghum, crambe, sinapsis alba, castor bean, sesame, cottonseed, linseed, soybean, arabidopsis, phaseolus, peanut, alfalfa, oat, rapeseed, barley, oat, rye, millet, dhurra, triticale, einkorn wheat, spelt, emmer wheat, flax, gramma grass, tripsacum, teosinte, festuca, perennial wheatgrass, sugarcane, cranberry, papaya, banana, safflower, oil palm, melon, apple, cucumber, dendrobium, gladiolus, chrysanthemum, Liliaceae, cotton, eucalyptus, sunflower, brassica, sugarbeet, coffee, ornamental plants, pines, etc. Preferably, the plant includes maize, soybean, mustard, millet, wheat, barley, rye, rice, cotton, and sorghum.

Optionally, the embodiments of the present application further provide use of any one of the above methods for improving agronomic traits of a plant in plant breeding. The plant includes any one of the above plants.

Optionally, the present application provides a plant cell, tissue, organ or commercial product which is not used as a propagation material, where the plant cell, tissue, organ or commercial product is obtained by the method of any one of claims 1 to 6. The commercial product includes, but is not limited to, whole or processed seeds, animal feed, oil, coarse powder, fine powder, flakes, bran, biomass or fuel products, etc.

Optionally, the embodiments of the present application further provide use of an expression cassette, recombinant vector or transformed strain in improving agronomic traits of a plant, which expression cassette, recombinant vector or transformed strain contains an isolated nucleotide sequence of a functional gene enabling the plant to acquire a salt-tolerant, alkali-tolerant and/or low nitrogen-tolerant phenotype, where the functional gene has an isolated nucleotide sequence selected from one of the sequences in the following group:
(a) a nucleotide sequence as shown in a gene position number of Zm00001d015410;
(b) a nucleotide sequence as shown in SEQ ID NO: 1 or 2;
(c) a nucleotide sequence encoding an amino acid sequence as shown in SEQ ID NO: 3;
(d) a nucleotide sequence capable of hybridizing with the nucleotide sequence in the sequences of (a) to (c) under a stringent condition;
(e) a nucleotide sequence having 80% (preferably at least 85%) sequence similarity with any one of the nucleotide sequences of (a) to (d), and enabling a plant to acquire a salt-tolerant, alkali-tolerant and/or low nitrogen-tolerant phenotype after being overexpressed in the plant; or
(f) a nucleotide sequence complementary to any one of the nucleotide sequences of (a) to (e).

Optionally, the expression cassette, recombinant vector or transformed strain further includes a promoter operably linked to the nucleotide sequence of the functional gene and driving excessive expression of the functional gene. The promoter includes, but is not limited to, the constitutive expression promoter, the specific expression promoter or the inducible expression promoter described above.

To avoid ambiguity, transferring, or introducing, or transforming a nucleotide sequence, a vector, a construct or an expression cassette into a plant described in the present application all means transferring a target nucleotide sequence, a construct, a vector or an expression cassette into a receptor cell or a receptor plant by a conventional transgenic method or a method of hybridization with the target transgenic plant. Any transgenic methods known to those skilled in the art can be used to transform recombinant expression vectors into plant cells to produce transgenic plants of the present application. Transformation methods can include direct or indirect transformation methods. Specifically, the transformation methods include but not limited to polyethylene glycol-induced DNA uptake, liposome-mediated transformation, gene gun introduction, electroporation, microinjection, the agrobacterium-mediated plant transformation method, etc.

Compared with the related art, the present application has the following beneficial effects:
(1) The present application provides a functional gene ZmSPL12 that regulates salt tolerance, alkali tolerance, and/or low nitrogen tolerance of a plant. The gene offers new genetic resources for stress-resistant breeding in a plant, especially crops such as maize, soybean, mustard, millet, wheat, barley, rye, rice, cotton, and sorghum.
(2) The present application provides a method for improving agronomic traits of a plant by introducing an over-expressed construct of a functional gene into the plant, which enables the plant to acquire a salt-tolerant, alkali-tolerant and/or low nitrogen-tolerant phenotype, provides the possibility for cultivating new crop varieties, especially new maize varieties, with salt-alkali tolerance and low nitrogen tolerance, and is of great significance to global food security and sustainable agricultural development.

### BRIEF DESCRIPTION OF THE DRAWINGS

FIGS. 1A-1D show the effects of salt and alkali treatments on the transcription level of ZmSPL12; where FIG. 1A shows the change in ZmSPL12 expression level in leaves at different time periods after treatment with 150 mM NaCl; FIG. 1B shows the change in ZmSPL12 expression level in roots at different time periods after treatment with 150 mM NaCl; FIG. 1C shows the change in ZmSPL12 expression level in leaves at different time periods after treatment with 100 mM NaHCO₃; FIG. 1D shows the change in ZmSPL12 expression level in roots at different time periods after treatment with 100 mM NaHCO₃.

FIGS. 2A-2F show the effects of treatments with different concentrations of nitrate on the transcription level of ZmSPL12; where FIG. 2A shows the change in ZmSPL12 expression level in leaves at different time periods after treatment with 0.8 mM KNO₃; FIG. 2B shows the change in ZmSPL12 expression level in roots at different time periods after treatment with 0.8 mM KNO₃; FIG. 2C shows the change in ZmSPL12 expression level in leaves at different time periods after treatment with 3.2 mM KNO₃; FIG. 2D shows the change in ZmSPL12 expression level in roots at different time periods after treatment with 3.2 mM KNO₃; FIG. 2E shows the change in ZmSPL12 expression level in leaves at different time periods after treatment with 14.4 mM KNO₃; FIG. 2F shows the change in ZmSPL12 expression level in roots at different time periods after treatment with 14.4 mM KNO₃.

FIGS. 3A-3C show the obtaining of a ZmSPL12 homozygous mutant and an overexpression material; where FIG. 3A is a schematic diagram of a CRISPR/Cas9 vector for ZmSPL12; FIG. 3B is the sequence analysis of a ZmSPL12 knockout mutant, where the target 1 sequence in WT is shown in SEQ ID NO: 6, the target 2 sequence in WT is shown in SEQ ID NO: 7, the target 1 sequence in KO#1 is shown in SEQ ID NO: 8, the target 2 sequence in KO#1 is shown in SEQ ID NO: 9, the target 1 sequence in KO#2 is shown in SEQ ID NO: 10, and the target 2 sequence in KO#2 is shown in SEQ ID NO: 11; FIG. 3C is a schematic diagram of an overexpression vector for ZmSPL12.

FIGS. 4A-4F show the phenotypes and phenotypic analysis of wild-type maize and ZmSPL12 overexpression transgenic lines grown under salt stress conditions; where FIG. 4A shows the phenotypes of the ZmSPL12 overexpression materials under salt stress; FIGS. 4B-4F show the determinations of relevant indicators of the ZmSPL12 overexpression materials under salt stress.

FIGS. 5A-5F show the phenotypes and phenotypic analysis of wild-type maize and ZmSPL12 overexpression transgenic lines grown under alkali stress conditions; where FIG. 5A shows the phenotypes of the ZmSPL12 overexpression materials under alkali stress; FIGS. 5B-5F show the determinations of relevant indicators of the ZmSPL12 overexpression materials under alkali stress

FIGS. 6A-6F show the changes in sodium and potassium ion contents in seedling leaves of wild-type maize and ZmSPL12 knockout lines grown under normal conditions and salt stress conditions; where FIGS. 6A-6C show the changes of sodium and potassium ion contents and the ratio of sodium ions to potassium ions of the aboveground part in different ZmSPL12 materials under salt stress; FIGS. 6D-6F show the changes of sodium and potassium ion contents and the ratio of sodium ions to potassium ions in the underground part of different ZmSPL12 materials under salt stress.

FIGS. 7A-7B show the changes in hydrogen peroxide and superoxide anion level in seedling leaves of wild-type maize and overexpression transgenic lines grown in salt-containing culture solution; where FIG. 7A shows the change of H₂O₂ level in different ZmSPL12 materials under salt stress; FIG. 7B shows the change of O^{2·-} level in different ZmSPL12 materials under salt stress.

FIGS. 8A-8G show the phenotypes and phenotypic analysis of wild-type maize, ZmSPL12 knockout lines and overexpression transgenic lines grown under low nitrogen conditions; where FIG. 8A shows the phenotypes of the ZmSPL12 knockout lines and overexpression transgenic lines under low nitrogen conditions; FIGS. 8B-8G show the determinations of relevant indicators of the knockout lines and overexpression transgenic lines under low nitrogen conditions.

FIGS. 9A-9F show the contents of different forms of nitrogen in wild-type maize and ZmSPL12 overexpression transgenic lines under low nitrogen conditions; where FIGS. 9A-9C show the contents of nitrate (NO₃⁻), nitrite (NO₂⁻) and ammonium (NH₄⁺) in the aboveground part of the ZmSPL12 overexpression transgenic lines under low nitrogen conditions; and FIGS. 9D-9F show the contents of nitrate (NO₃⁻), nitrite (NO₂⁻) and ammonium (NH₄⁺) in the underground part of the ZmSPL12 overexpression transgenic lines under low nitrogen conditions.

### DETAILED DESCRIPTION OF THE EMBODIMENTS

In order to facilitate the understanding of the present application, a more comprehensive description of the present application will be provided below. However, the present application can be implemented in many different forms and is not limited to the embodiments described herein. Conversely, the purpose of providing these embodiments is to enable a more thorough and comprehensive understanding of the disclosure of the present application.

The experimental methods used in the following embodiments are conventional methods unless otherwise specified; the materials, reagents, etc. used are all commercially available unless otherwise specified; quantitative experiments are conducted with more than 5 replicates unless otherwise specified.

### Embodiment 1

### ZmSPL12 Involved in Response of Plant to Salt and Alkali Stress

The position of ZmSPL12 gene described in the present application on maize genome is Zm00001d015410, which has a genome nucleotide sequence as shown in SEQ ID NO: 1, an encoding region (CDS) nucleotide sequence as shown in SEQ ID NO: 2, and an encoded protein amino acid sequence as shown in SEQ ID NO: 3.

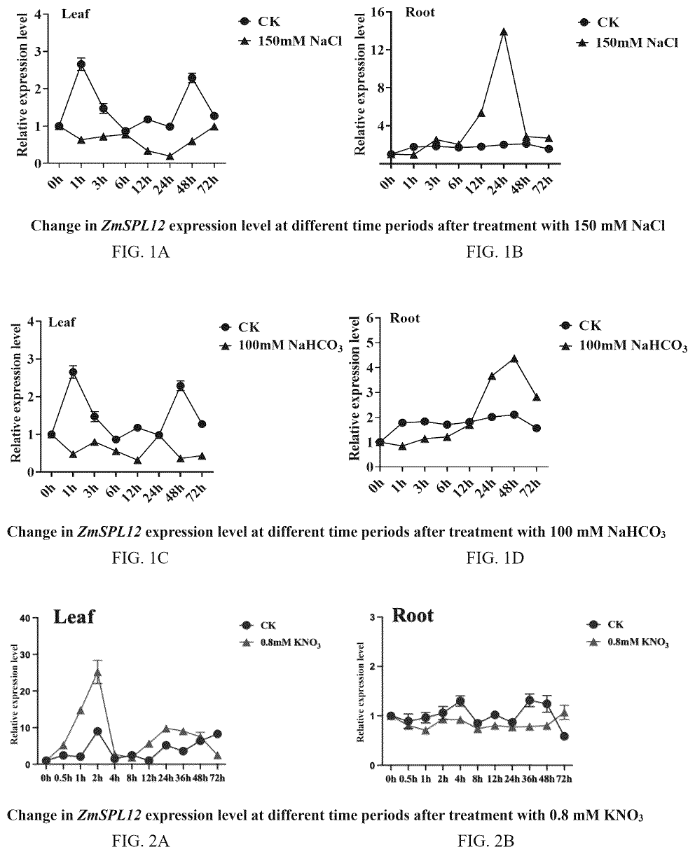

To verify whether the expression of ZmSPL12 is involved in the response of a plant to salt and alkali stress, C01 maize materials at the two-leaf and one-heart stage were placed in a 150 mM NaCl aqueous solution and a 120 mM NaHCO₃ aqueous solution, with one group placed in water set as their control, respectively. Young leaves and roots with consistent growth status were collected at 0 h, 1 h, 3 h, 6 h, 12 h, 24 h, 48 h, and 72 h after salt and alkali treatments, along with their corresponding controls, respectively, for fluorescent quantitative PCR experiment on the ZmSPL12 gene. The specific primers were: ZmSPL12-qPCR-F 5′-GATGGAGCAGCGGATCTTCA-3′ (SEQ ID NO: 4), ZmSPL12-qPCR-R 5′-AGTTCCTGCCATGCGTTACA-3′ (SEQ ID NO: 5). The results show that under salt stress, the expression level of ZmSPL12 in leaves is downregulated, but significantly upregulated in roots, reaching its peak at 24 h (FIG. 1A and FIG. 1B); under alkali stress, the expression level of ZmSPL12 in leaves is downregulated, but significantly upregulated in roots, reaching its peak at 48 h (FIG. 1C and FIG. 1D). The results indicate that the expression of ZmSPL12 in roots of maize seedlings is induced by salt and alkali signals, suggesting that ZmSPL12 may be involved in the response of a plant to salt and alkali stress.

### Embodiment 2

### ZmSPL12 Involved in Response of Plant to Nitrate Signal

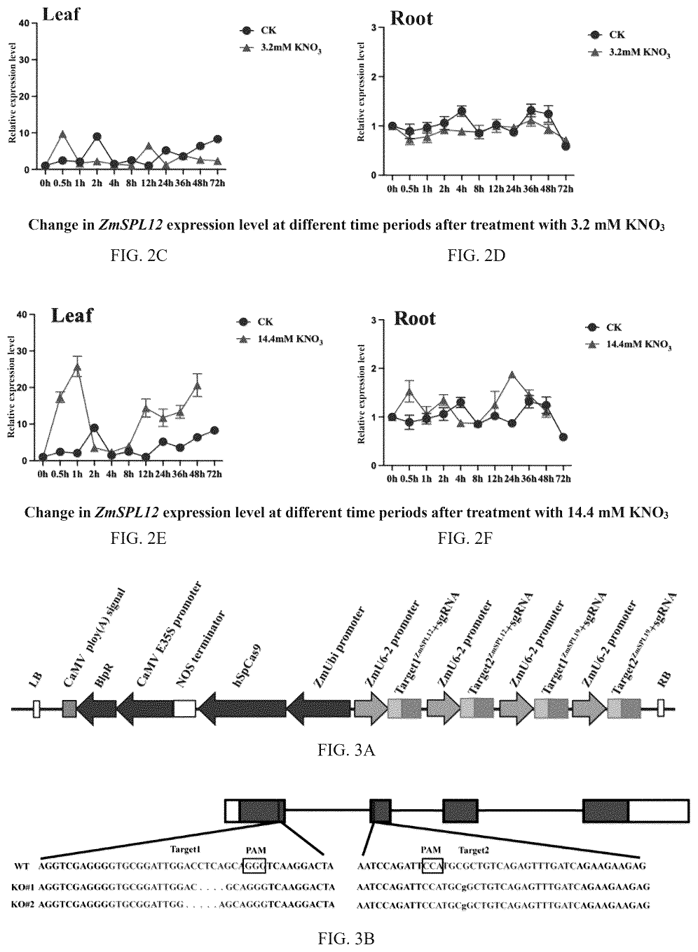

To verify whether the expression of ZmSPL12 is involved in the response of a plant to nitrate signal, B73 maize materials at the two-leaf and one-heart stage were placed in a nutrient solution containing 0.8 mM KNO₃, a nutrient solution containing 3.2 mM KNO₃, and a nutrient solution containing 14.4 mM KNO: , with one group placed in a nutrient solution without KNO₃ set as their controls, respectively. The composition of the nutrient solution is (mmol/L): K₂SO₄ 0.75, KH₂PO₄ 0.25, MgSO₄·7H₂O 0.65, DETA-NaFe 0.1, HaBO₃ 0.01, MnSO₄·H₂O 0.001, ZnSO₄·7H₂O 0.001, CuSO₄ 5H₂O 0.0001, (NH₄)₆Mo₇O₂₄·4H₂O 0.5×10⁶, and CaCl₂ 0.2. Young leaves and roots with consistent growth status were collected at 0 h, 0.5 h, 1 h, 2 h, 4 h, 8 h, 12 h, 24 h, 36 h, 48 h, and 72 h after the treatment, along with their corresponding controls, respectively, for fluorescent quantitative PCR experiment on the ZmSPL12 gene. The results show that under treatment with 0.8 mM KNO₃, the expression level of ZmSPL12 in leaves is significantly upregulated, reaching its peak at 2 h, but not significantly changed in roots (FIG. 2A and FIG. 2B); under treatment with 3.2 mM KNO₃, the expression level of ZmSPL12 in leaves is significantly upregulated, reaching its peak at 0.5 h, but not significantly changed in roots (FIG. 2C and FIG. 2D); under treatment with 14.4 mM KNO₃, the expression level of ZmSPL12 in leaves is significantly upregulated, reaching its peak at 1 h, but rises with fluctuation in roots, reaching its peak at 24 h (FIG. 2E and FIG. 2F). The results indicate that the expression of ZmSPL12 in roots of maize seedlings is induced by nitrate signal, suggesting that ZmSPL12 may be involved in the response of a plant to nitrate signal.

### Embodiment 3

### Obtaining of ZmSPL12 Homozygous Mutant and Overexpression Material

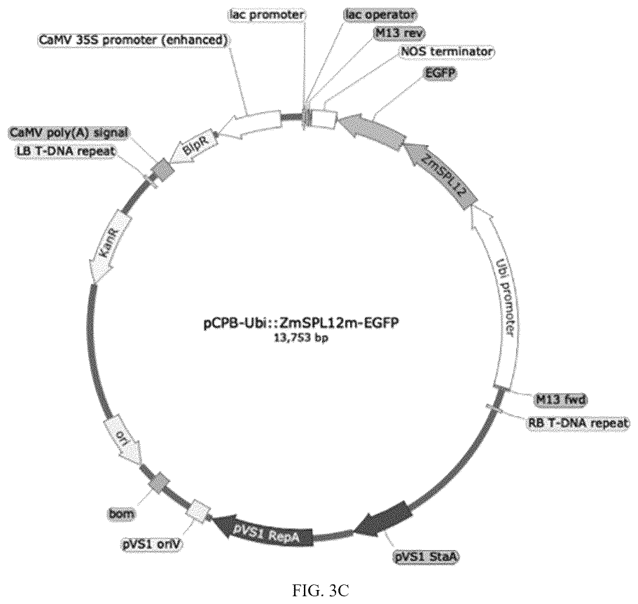

To study the function of the ZmSPL12 gene, the inventors constructed a CRISPR/Cas9 gene knockout vector for ZmSPL12 and performed genetic transformation on maize plants, resulting in a total of 10 effective independent transformation events. Where, the structure of the CRISPR/Cas9 gene editing vector is as shown in FIG. 3A. Through PCR identification and Sanger sequencing of nucleotide sequences, homozygous mutants of Zmspl12 (K #1 and KO#2) were ultimately obtained. The target sequences for the gene editing and the mutation sites are as shown in FIG. 3B, respectively. Where, the mutation type of Zmspl12-KO#1 is a frameshift mutation caused by 4 bp deletion at the first target site; the mutation type of Zmspl12-KO#2 is a frameshift mutation caused by a 5 bp deletion at the first target site. In addition, both mutants include a single-base insertion at the second target site.

Meanwhile, the inventors also constructed a ZmSPL12 overexpression vector pCPB-Ubi::ZmSPL12m-EGFP, with the vector structure as shown in FIG. 3C. And agrobacterium-mediated transformation was performed on maize plants to obtain homozygous ZmSPL12-OE overexpression materials (with the C01 background) after transgenic screening. The two overexpression lines identified as having increased expression level were designated as #499 and #500, respectively.

### Embodiment 4

### Phenotypes of ZmSPL12 Overexpression Materials Under Salt Stress

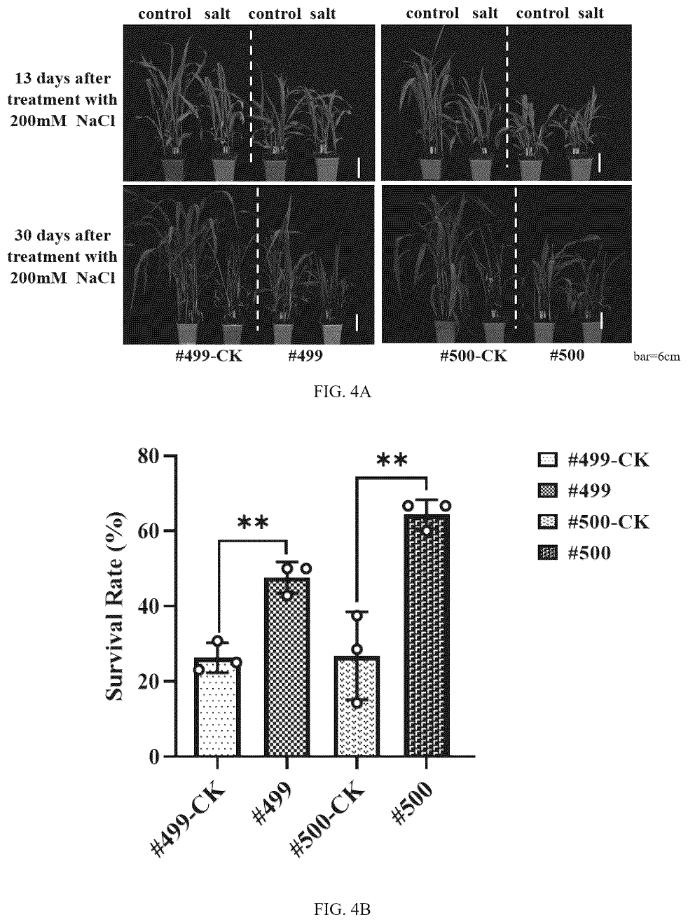

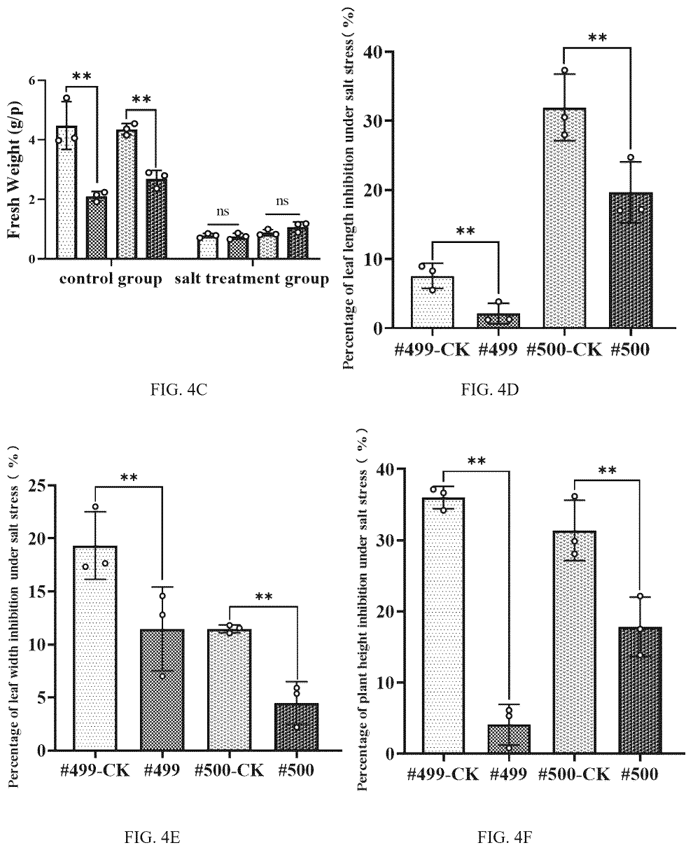

To verify whether ZmSPL12 is involved in the response to salt stress, the inventors subjected the ZmSPL12-OE materials identified as having increased expression levels and their corresponding control materials to salt stress treatment. The results indicate that after 13 days of treatment, the ZmSPL12 overexpression materials #499 and #500 grow normally, while the first leaves of #499-CK and #500-CK wither; after 30 days of treatment, the survival rates of the overexpression materials #499 and #500 are significantly higher than those of #499-CK and #500-CK; the fresh weights of the overexpression materials #499 and #500 in the control groups are significantly lower than those of #499-CK and #500-CK, while fresh weights of the overexpression materials in the treatment groups show no significant difference compared to those of their corresponding control materials; the stress ratios of leaf length/leaf width/plant height in the overexpression materials #499 and #500 are significantly lower than those of #499-CK and #500-CK (FIGS. 4A-4F). The results demonstrate that the salt tolerance of maize is enhanced after overexpression of the ZmSPL12 gene.

### Embodiment 5

### Phenotypes of Different ZmSPL12 Materials Under Alkali Stress

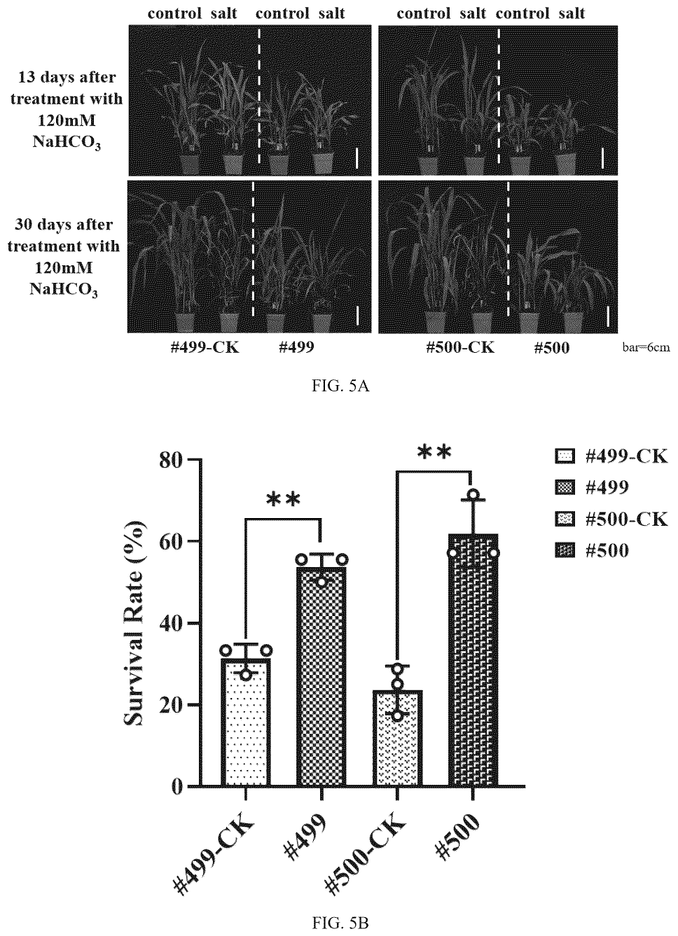

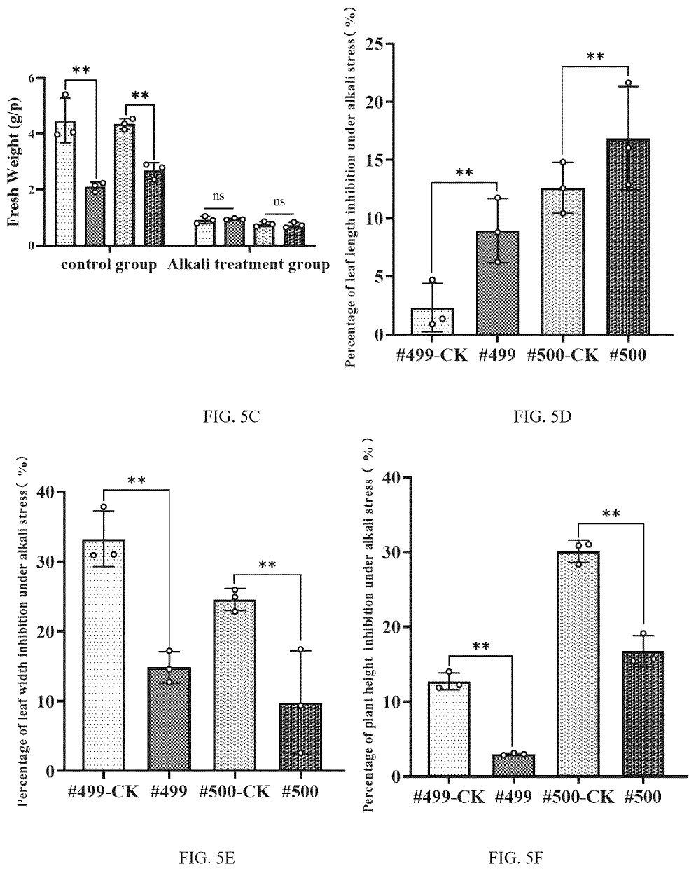

To verify whether ZmSPL12 is involved in the response to alkali stress, the inventors subjected the ZmSPL12-OE materials identified as having increased expression level and their corresponding control materials to alkali stress treatment. The results indicate that after 13 days of treatment, the ZmSPL12 overexpression materials #499 and #500 grow normally, while the first leaves of #499-CK and #500-CK wither; after 30 days of treatment, the survival rates of the overexpression materials #499 and #500 are significantly higher than those of #499-CK and #500-CK; the fresh weights of the overexpression materials #499 and #500 in the control groups are significantly lower than those of #499-CK and #500-CK, while fresh weights of the overexpression materials in the treatment groups show no significant difference compared to those of their corresponding control materials; the stress ratios of leaf width/plant height in the overexpression materials #499 and #500 are significantly lower than those of #499-CK and #500-CK, while the stress ratios of leaf length are on the contrary (FIGS. 5A-5F). The results demonstrate that overexpression of the ZmSPL12 gene enhances the alkali tolerance of maize; compared to salt stress, alkali stress has a greater effect on plant leaf width but a reduced effect on plant height and leaf length, indicating that there may be different molecular mechanisms underlying the response of a plant to salt and alkali stresses.

### Embodiment 6

### Determination of Sodium and Potassium Ion Contents in Different ZmSPL12 Materials

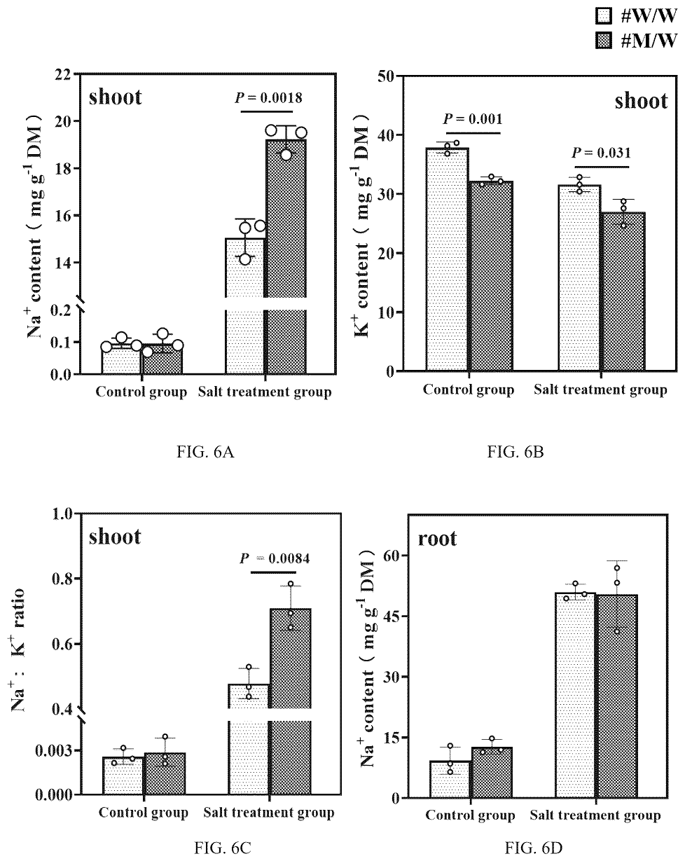

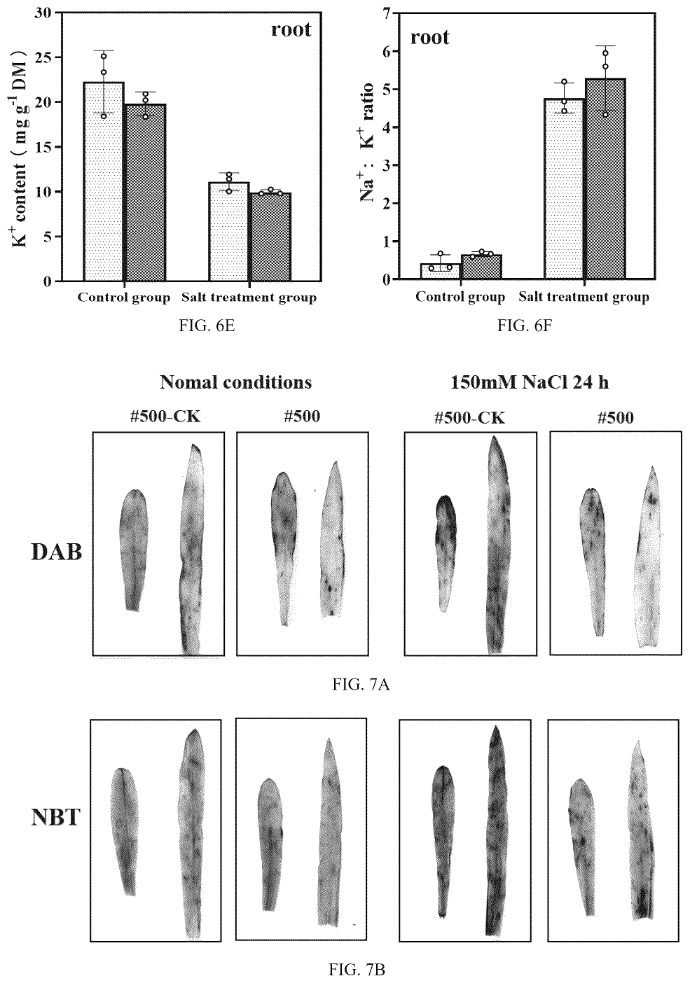

Furthermore, the sodium and potassium ion contents in the materials under normal and salt treatment conditions were determined in the present application. The results indicate that under salt stress, the aboveground part tissues of ZmSPL12 knockout plants exhibit a higher Na⁺ content, a lower K⁺ content, and a higher Na⁺/K⁺ ratio; the sodium and potassium ion contents of the underground part tissues show no significant difference compared to those of their controls (FIGS. 6A-6F). The results demonstrate that ZmSPL12 enhances the salt tolerance of a plant by promoting ion (Na⁺ and K⁺) homeostasis.

### Embodiment 7

### Detection of ROS Level in Different ZmSPL12 Materials Under Salt Stress

The inventors used DAB and NBT staining to qualitatively detect the levels of H₂O₂ and O^{2·-} in a plant under control and salt stress conditions. As shown in FIGS. 7A-7B, under control conditions, the levels of H₂O₂ and O^{2·-} accumulated in different ZmSPL12 materials are approximately comparable, while under salt stress conditions, the levels of H₂O₂ and O^{2·-} accumulated in ZmSPL12-OE materials are significantly lower than those in their control materials. The results demonstrate that ZmSPL12 is involved in the regulation of in vivo ROS level under salt stress, and excessive expression of ZmSPL12 can significantly reduce the in vivo ROS content in a plant.

### Embodiment 8

### Phenotypes of ZmSPL12 Knockout and Overexpression Materials Under Low Nitrogen Treatment

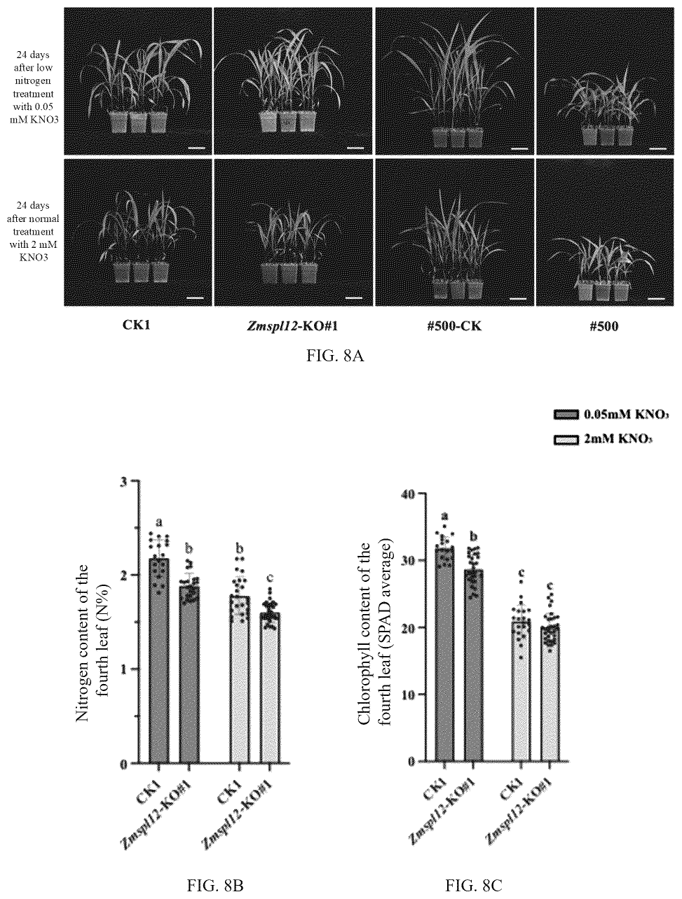

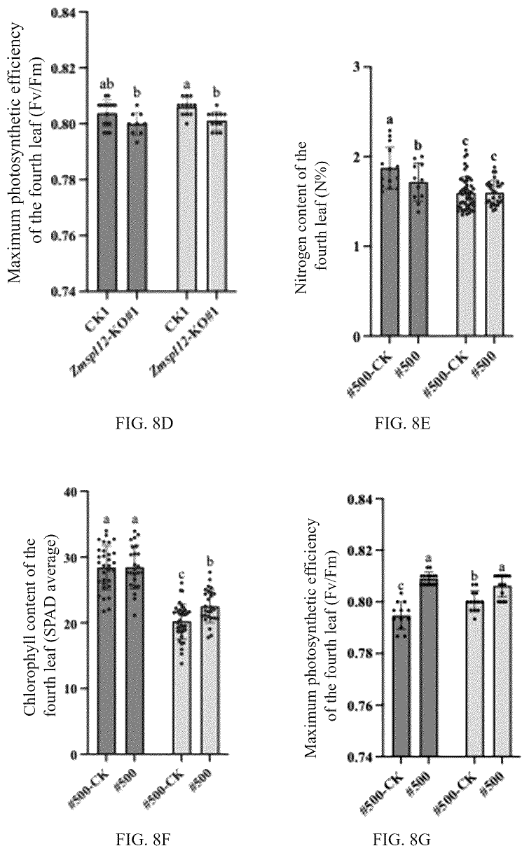

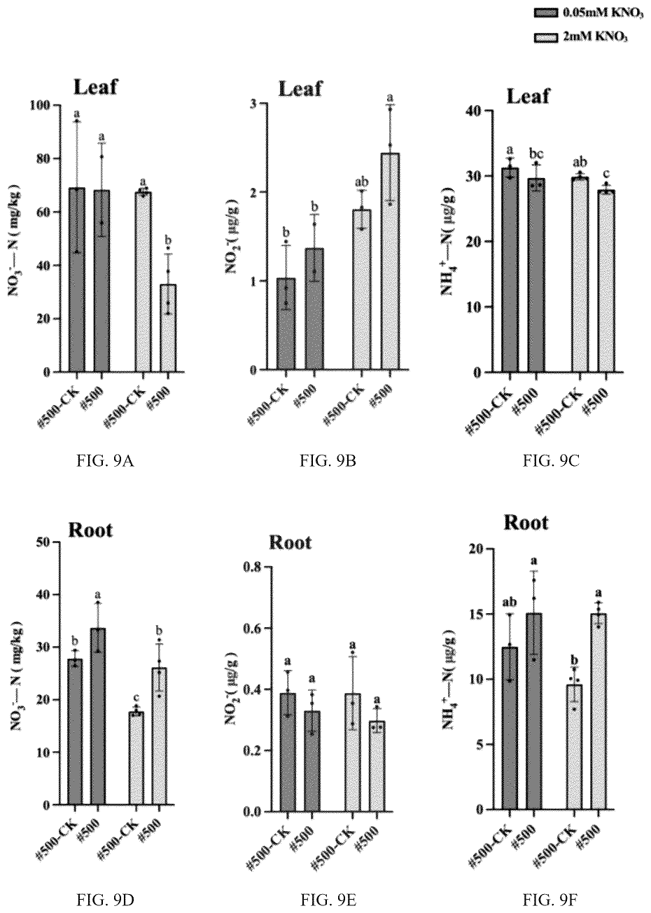

To verify whether ZmSPL12 enhances the tolerance to low nitrogen environment, the inventors subjected ZmSPL12 knockout materials and the ZmSPL12-OE materials identified as having increased expression level to low nitrogen treatment (which is 0.05 mM KNO₃, with 2 mM KNO₃ as the normal control). The typical characteristic of nitrogen deficiency in a plant is the decrease in leaf nitrogen content and chlorophyll content. The results indicate that after 24 days of treatment, the first leaf of the ZmSPL12 knockout plant Zmspl12-KO#1 under low nitrogen treatment withers, and the second leaf turns yellow, while only the first leaf of the wild-type control CK1 withers; the phenotype of the ZmSPL12 overexpression material is just on the contrary: #500 grows normally, while the first leaf of the wild-type control #500-CK withers and the second leaf turns yellow; under low nitrogen treatment, the nitrogen content of the fourth leaf in Zmspl12-KO#1 is significantly lower than that in the wild-type control CK1, but the nitrogen content of the fourth leaf in #500 shows no significant difference compared to that in the wild-type control #500-CK; under both normal and low nitrogen conditions, the chlorophyll content of the fourth leaf in Zmspl12-KO#1 is lower than that in the wild-type control CK1, while the chlorophyll content of the fourth leaf in #500 shows no significant difference compared to that in the wild-type control #500-CK under normal conditions, but is significantly higher than that in the wild-type under low nitrogen conditions; under both normal and low nitrogen conditions, the maximum photosynthetic efficiency of the fourth leaf in Zmspl12-KO#1 is lower than that in the wild-type control CK1, with a significant difference observed under low nitrogen conditions in particular, while #500 is just on the contrary, which exhibits a significantly higher maximum photosynthetic efficiency of the fourth leaf compared to that in the wild-type control #500-CK under both normal and low nitrogen treatment conditions (FIGS. 8A-8G).

The results demonstrate that the knockout of the ZmSPL12 gene reduces the tolerance of maize to low nitrogen conditions. On the contrary, the overexpression of the ZmSPL12 gene enhances the tolerance of maize to low nitrogen conditions, further demonstrating that the ZmSPL12 gene increase the nitrogen use efficiency of the plant.

### Embodiment 9

### Detection of Contents of Different Forms of Nitrogen in ZmSPL12 Overexpression Material Under Low Nitrogen Treatment

Furthermore, the present application determined the contents of nitrate (NO₃⁻), nitrite (NO₂⁻) and ammonium (NH₄⁺) in the ZmSPL12 overexpression material #500 and its wild-type control #500-CK under normal and low nitrogen treatment conditions, respectively. The results indicate that: in the leaves of the aboveground part, under normal treatment conditions, the contents of nitrate and nitrite in #500 show no significant difference compared to those in the wild-type control #500-CK, while the ammonium content in #500 is significantly lower than that in the wild-type control #500-CK. Under low nitrogen treatment conditions, the contents of nitrate and ammonium in #500 are both significantly lower than those in the wild-type control #500-CK, while the content of nitrite in #500 shows no significant difference compared to that in the wild-type control #500-CK; in the roots of the underground part, under normal treatment conditions, the contents of nitrite and ammonium in #500 show no significant difference compared to those in the wild-type control #500-CK, but the nitrate content in #500 is significantly higher than that in the wild-type control #500-CK. Under low nitrogen treatment conditions, the content of nitrite in #500 shows no significant difference compared to that in the wild-type control #500-CK, but the contents of nitrate and ammonium in #500 are significantly higher than those in the wild-type control #500-CK. The results demonstrate that ZmSPL12 enhances nitrogen use efficiency of a plant by enhancing the contents of nitrate and ammonium in roots, thereby enhancing the tolerance to low nitrogen environments.

The embodiments described above merely represent several embodiments of the present application, which are described in a relatively specific and detailed manner, but shall not be construed as limiting the scope of the patent in the present application. It should be pointed out that for those of ordinary skill in the art, several modifications and improvements can be made without departing from the concept of the present application, and these all fall within the protection scope of the present application. Therefore, the protection scope of the patent in the present application shall be subject to the appended claims.

1. A method for improving agronomic traits of maize by introducing an over-expressed construct of a functional gene into a maize plant to enable the maize plant to acquire a salt-tolerant and/or alkali-tolerant phenotype, wherein the functional gene has a nucleotide sequence selected from one of sequences in the following group:
(a) the nucleotide sequence as shown in SEQ ID NO: 2;
(b) a nucleotide sequence encoding the amino acid sequence as shown in SEQ ID NO: 3;
(c) a nucleotide sequence complementary to any one of the nucleotide sequences of (a) to (b).

2. The method according to claim 1, wherein the over-expressed construct further comprises a promoter operably linked to the nucleotide sequence of the functional gene and driving excessive expression of the functional gene.

3. The method according to claim 2, wherein the promoter is a cauliflower mosaic virus CaMV 35S promoter, a maize ubiquitin Ubiquitin promoter, or a rice Actin1 promoter.

4. A use of the method according to any one of claims 1 to 3 in maize breeding.


SEQUENCE LISTING (11 sequences)

SEQ ID NO: 1 - DNA, 3504 bp, Zea mays
  source 1..3504: mol_type = genomic DNA
catctcgtcttctccacagtccacactgttcaccacctctcccatcctctcttcgatccccaaatcccgaattgaccagcccaaatcgtcgccgcggcgcatggagtggacggccccgaagcccgcctcatcgcctcccctcctctgggactggggagaccacgccgccacgggctcgggctcctccagcgacgccccggcgcggcgcggcgggaaggagcgggaggcgaagcgtgccagggccggcgaggaccgcgggggaggtgagctgaggtgccaggtcgaggggtgcggattggacctcagcagggtcaaggactaccaccgtaagcaccgcgtctgcgaggcccacaccaagtccccccgcgtcatcgtcgccggccaggagcgccggttctgccagcagtgcagccggtgggtcgtcttctcgcttgctctcgcttgcctcctccacgcctcttctctcgcttgctctgtgcgattgcttccgtcttccttcctttgattgctttctgcaacgatggttcgcgcttttgcgctgcaattatcgcgtgatttgttgattggtttggacaagcagttgttttttacgtgttctctattagttttgtggctacatgctgtgtgtgtgtacggcgcgtgagctgcgtgcgcggttgtgccgtttttttgggtacggaactacatttttttcctgatctagcaatgaaagatgcttcctttccactattggtactgttgtacttgttgtggagaatcgtacgcatgggtgaaatgagaaatggctactatgcctgtttcccttttccagttttggtatggctctcgatatatgggtatgcttttgggatgagcgtttttttgtgggctactgacccgtgtctattttattccattaaaactatgtgaatattgcaggcatatatctcctgtattgcacttttacatggcatttaaccataaatagtgtctattttgtttttgctgattgcttgggttctggcaatccagattccatgcgctgtcagagtttgatcagaagaagaggagctgcaggaggcgtctgtctgatcacaatgcccgccgccggaagcctcagccagatgcattcaccttcgcctctgcaaagctgccttcgacattgtttggtaatgttttcatatcttgactactgttcgaaatttgctatcagtggaagtggacactagaaaaaatggtggtgatgggctcttagccaggttctagattcatctgcatgtacgtcatgcaaacagatgtcttgttacgcaaatggcaagctttctatcttttatacttagcaataagttgtctgttgtgtttcttcgtgtaattaaatagaaactaggcattggacttgagagtatcgtgattagtaatatatgtatcttgtactatacagctagcaggattatcgtgtttcaattgtattattctaccttcttacctcctatttataaagtgaaatattcttatccctgaacttctctgccactgcagatgataggcggcaaataagttttgtctggaataaagctcctgttagccatgtaagacccttcacttctccatgggacagctcatctgacttaaagcccccttatgcaaaggaaataagtgatgtatcaacaaaagttgggacaattactggacaagttcatttggataaatctcacatgttcaatgctattccaacacttagccatggcaaagatgagccttggccaatgaaaggtttgattttccaaacattatccatacttgctgcagaaatgtgttcatcaaggaagaagctttgttaagtggtttgccatatttgttgacaaagggttgtaaaccatctccggtttagtcggaccttacctaaaacatcttccgcagttgacacataaaagggacctttatccattttatcatcgcttgcagagatgggaagctgattattattacacgagtactttgaattttcattgcagaagcttgtgaacttaaaagtcaaaatatttttgtagctattcatatgatttagcctgcatagaatgtatggcggaacaaagattgctgaactccttgccatacctctattcaaactgggcttatagatgttttgttgtaggttgcttgattctttactctgtaaatttactaccagaaaaaagaagaatgctaccagaattgaactgtcagttgtattcacatgttagtttaggatgcaaacacttgattatgttggatttatgggaaataaaatgttgcagggactgataccattttctccaaaatggtattgggacttggttattagcgatgatggattttgagtgccaagaaaacgtagatgtttttctatcaatatcatatttggtagcattattttatttgtttagtttcacctattctaggtttctgaagtcctagatcactgttgagataattaattactccatgtttcgtttatggatgacatgtaaacttcattttgcttgcaagaaattactaataaactgcaggtctatttgactctcctgcacttacattttactgtgctagtgaccagtttaataattctgagcatcagagttagaaattgtaattgactgtcatacaactctgcttgttttcatgctagttttattgaaccatgtcatgtgatttcatttgccatgccgttcctgttcctgcaccgtctgcacttcgatgttcttttgttgtcccgatgggtgtatgacatagtctgaatgatcggttctacaggtctggacatgtctatatctgcttcaaaattcgatggagcagcggatcttcagcatgctctctctcttctgtcagccggcgcttgtggactgcctgattctgtacatcaaacatcttgcattatccaattcaatggtgccagcgagaacagcagtgaccttcatgtaacgcatggcaggaactctggtccagcatcatgcgccgatgcgcagcatatagctgcccagcctcagtctcagctgtttcattttaccacagataccggcaatactgtttatgagcccagtttctttggtgtaaaccagataaattaaccatggaagacacatgccacttgatcacttggatgaatagccctgaagatggtctgggcctgtgcaagttcttcaaccaaccaccggacataaggatctttgttaatgttatcttcatatttattggccatacctgaattcagttgaaccgagcttaggccagcttatgtttaatatgcagttccaacctgtgattgacccattttttatgttctttttgccctctgtgatgtgagtgtgactactctgtaacattataagtgtgatactattgtagtctctactgccctgacgatgtaaacggttggatatctttgaagctgtccttgatgaatctggacatgtattttgtgtgtatatatggtctgttgcttcaatactgccatcattaaaatatttgaaggacttcagctattgc

SEQ ID NO: 2 - DNA, 999 bp, Zea mays
  source 1..999: mol_type = other DNA
atggagtggacggccccgaagcccgcctcatcgcctcccctcctctgggactggggagaccacgccgccacgggctcgggctcctccagcgacgccccggcgcggcgcggcgggaaggagcgggaggcgaagcgtgccagggccggcgaggaccgcgggggaggtgagctgaggtgccaggtcgaggggtgcggattggacctcagcagggtcaaggactaccaccgtaagcaccgcgtctgcgaggcccacaccaagtccccccgcgtcatcgtcgccggccaggagcgccggttctgccagcagtgcagccgattccatgcgctgtcagagtttgatcagaagaagaggagctgcaggaggcgtctgtctgatcacaatgcccgccgccggaagcctcagccagatgcattcaccttcgcctctgcaaagctgccttcgacattgtttgatgataggcggcaaataagttttgtctggaataaagctcctgttagccatgtaagacccttcacttctccatgggacagctcatctgacttaaagcccccttatgcaaaggaaataagtgatgtatcaacaaaagttgggacaattactggacaagttcatttggataaatctcacatgttcaatgctattccaacacttagccatggcaaagatgagccttggccaatgaaaggtctggacatgtctatatctgcttcaaaattcgatggagcagcggatcttcagcatgctctctctcttctgtcagccggcgcttgtggactgcctgattctgtacatcaaacatcttgcattatccaattcaatggtgccagcgagaacagcagtgaccttcatgtaacgcatggcaggaactctggtccagcatcatgcgccgatgcgcagcatatagctgcccagcctcagtctcagctgtttcattttaccacagataccggcaatactgtttatgagcccagtttctttggtgtaaaccagataaattaa

SEQ ID NO: 3 - protein, 332 aa, Zea mays
MEWTAPKPASSPPLLWDWGDHAATGSGSSSDAPARRGGKEREAKRARAGEDRGGGELRCQVEGCGLDLSRVKDYHRKHRVCEAHTKSPRVIVAGQERRFCQQCSRFHALSEFDQKKRSCRRRLSDHNARRRKPQPDAFTFASAKLPSTLFDDRRQISFVWNKAPVSHVRPFTSPWDSSSDLKPPYAKEISDVSTKVGTITGQVHLDKSHMFNAIPTLSHGKDEPWPMKGLDMSISASKFDGAADLQHALSLLSAGACGLPDSVHQTSCIIQFNGASENSSDLHVTHGRNSGPASCADAQHIAAQPQSQLFHFTTDTGNTVYEPSFFGVNQIN

SEQ ID NO: 4 - DNA, 20 bp, synthetic construct
  source 1..20: mol_type = other DNA
gatggagcagcggatcttca

SEQ ID NO: 5 - DNA, 20 bp, Synthetic construct
  source 1..20: mol_type = other DNA
agttcctgccatgcgttaca

SEQ ID NO: 6 - DNA, 43 bp, synthetic construct
  source 1..43: mol_type = other DNA
aggtcgaggggtgcggattggacctcagcagggtcaaggacta

SEQ ID NO: 7 - DNA, 43 bp, synthetic construct
  source 1..43: mol_type = other DNA
aatccagattccatgcgctgtcagagtttgatcagaagaagag

SEQ ID NO: 8 - DNA, 39 bp, synthetic construct
  source 1..39: mol_type = other DNA
aggtcgaggggtgcggattggacgcagggtcaaggacta

SEQ ID NO: 9 - DNA, 44 bp, synthetic construct
  source 1..44: mol_type = other DNA
aatccagattccatgcggctgtcagagtttgatcagaagaagag

SEQ ID NO: 10 - DNA, 38 bp, synthetic construct
  source 1..38: mol_type = other DNA
aggtcgaggggtgcggattggagcagggtcaaggacta

SEQ ID NO: 11 - DNA, 44 bp, synthetic construct; identical to SEQ ID NO: 9
  source 1..44: mol_type = other DNA